(19) United States
(12) Patent Application Publication
(10) Pub. No.: US 2023/0255475 A1
(43) Pub. Date: Aug. 17, 2023

(54) REFLECTION BASED CORNEAL TOPOGRAPHY SYSTEM USING PRISMS FOR IMPROVED ACCURACY AND METHOD OF USE

(71) Applicant: Paul Phillip VAN SAARLOOS, TANJUNG BUNGAH (MY)

(72) Inventor: Paul Phillip VAN SAARLOOS, TANJUNG BUNGAH (MY)

(21) Appl. No.: 18/012,380

(22) PCT Filed: Jul. 5, 2021
(86) PCT No.: PCT/MY2021/050055

§ 371 Date: Dec. 22, 2022

(30) Foreign Application Priority Data
Jul. 21, 2020 (MY) PI2020003761

(51) Int. Cl. A61B 3/107; A61B 3/15

(52) U.S. Cl. CPC A61B 3/107; A61B 3/152

(57) ABSTRACT

Provided herein is a corneal topography system (218) that utilizes a prism placed in optical alignment between the pattern generator (201), such as a Placido disk, and the eye. The corneal topography system may be a prismatic triangulating corneal topography system that utilizes light rays of angle θ at the edge of the prism not passing through the prism (202), and using the deviation of the light rays passing through the prism at that edge to calculate angle θ. With angle α calculated from the reflected image on the image sensor (209) intersecting with the light ray from the pattern generator (201) at angle θ at the reflection point on the corneal surface (207). This provides both the position and slope of the corneal surface (207) at that point. Also provided is a method for mapping a corneal surface of an eye of a subject utilizing an optical prism (202) to produce a reflection image from a corneal surface reflection point (206) on the corneal surface (207) of the eye.

### FIELD OF THE INVENTION

This invention relates to ophthalmic instruments. More specifically, the present invention is directed to an improved system and method for measuring the anterior corneal surface curvature and topography by capturing Placido disk images reflected in the cornea.

### BACKGROUND OF THE INVENTION

Measuring the topography of the corneal surface has become an important part of ophthalmology. It is used to detect disorders like keratoconus and to assess an eye's suitability for and to customize surgery, for example, LASIK, or cataract surgery with the use of premium intra-ocular lenses.

There are currently two distinct technologies available for measuring corneal topography. One of them is the slit projection system, which involves projecting slits of light into the eye and capturing multiple images of the eye from a range of angles relative to the direction of the light slit projection, or different positions on the cornea. This technology includes Scheimpflug based systems. The second involves imaging targets reflected from the corneal surface. The target is usually a Placido disk which is made up of dark and light concentric rings.

Slit projection systems have the advantage that they can also measure the posterior surface of the cornea. However, the anterior surface of the cornea accounts of around two thirds of the focusing power of the eye, and there is currently debate as to whether the much lower optical effect of the posterior surface has any significant clinical relevance.

The slit projection method has some disadvantages. The eye can move during the multiple image captures using the different slit orientations/positions required to get the entire topography. This can lead to inaccurate measurements. In addition, because the reflection angle is double the angle of the surface doing the reflecting, and slit projection systems calculate topography using the sine of the angle between the slit project and viewing directions, the precision of slit projection systems is about a quarter that of reflection based systems, for a given camera resolution.

However, reflection based corneal topography systems also have disadvantages. Specifically, without prior knowledge on how far the corneal surface is from the topography device, there are many possible solutions for corneal topography. Three possible solutions are shown in FIG. 1A, i.e., a steep corneal surface closer to the topographer, a flat corneal surface further away from the device, and a medium curvature in between, could all result in the same reflected image.

Hence, it can be difficult to calculate the shape of the corneal surface from the image reflected from it, and without making assumptions about that shape there will not be a unique solution. One such method to calculate the corneal shape from the reflection image is described by van Saarloos et al. (1). van Saarloos discloses a mathematical method for estimating the central corneal radius of curvature and for calculating corneal topography from the radii of the rings in a Placido disk image. This method, as well as similar methods of calculation, require the distance between the eye and the topography device to be accurately known and for the eye shape to match the assumptions of shape used in the calculation methods.

Some topography systems rely on the user to accurately position the device relative to the eye, to ensure the distance from the device to the eye is known. These systems depend heavily on operator skills. Delays caused by the user failing to quickly get the device precisely lined up, can cause the eye to dry, creating an inaccurate measurement, and can increase operating costs.

U.S. Pat. No. 5,418,582 teaches a system and method where the Placido disk has an added ring or point light source well outside the plane formed by the adjacent rings. This allows parallax to be used to calculate the distance between the device and the eye and greatly simplifies use of the topographer since it removes user error. It allows calculation of angle θ from an image of the reflected rings, but only for one point or one ring. This improvement still relies on the assumptions of shape (corneal topography) for applying the calculations to the rest of the corneal surface.

Overall, therefore, there is a deficiency in the art for optimal topography systems and methods, which improve accuracy while limiting or eliminating the need for making assumptions of shape. The present invention fulfills this longstanding need and desire in the art.

### SUMMARY OF THE INVENTION

The present invention is directed to a corneal topography system for mapping a corneal surface of an eye in a subject. The system has at least a pattern generator and at least one optical prism in optical alignment between the pattern generator and the corneal surface of the eye for which topographical information is desired. A light source is positioned to illuminate the pattern generator and an image sensor is disposed in optical alignment with the corneal surface of the eye. A means for electronically transmitting data from the image sensor to an electronic device, which is configured to analyze the data and to display results of the analysis. The present invention is directed to a related corneal topography system further comprising a focusing lens placed between the image sensor and the corneal surface.

The present invention also is directed to a reflection based corneal topography system for mapping a corneal surface of an eye, that uses prisms to determine the direction the pattern generation target is from the reflection point on the cornea. The system has at least one Placido disk and at least one prism in optical alignment between the Placido disk and the corneal surface of the eye for which topographical information is desired. A light source is disposed to illuminate the Placido disk and an image sensor is disposed in optical alignment with the corneal surface of the eye. An electronic device comprising image analysis software tangibly stored therein is in electronic communication with the image sensor. The present invention is directed to a related reflection based corneal topography system with prisms further comprising an optical lens disposed between the image sensor and the corneal surface.

The present invention is directed further to a method for mapping a corneal surface of an eye of a subject. An optical prism is positioned between a Placido disk and the corneal surface of the eye of the subject in a corneal topography system and the Placido disk is illuminated to generate a ring pattern therefrom. A reflection of the illuminated Placido disk from the corneal surface of the eye is acquired with an image sensor, where part of the Placido disk image is deviated by refraction through the prism. The reflection image is transmitted from the image sensor to a computer to measure at least one parameter of the corneal surface and the at least one parameter is mapped to produce a corneal topography map of the eye. The present invention is directed to a related method for mapping a corneal surface of an eye of a subject further comprising displaying the corneal topography map on the computer.

Other and further aspects, features, benefits, and advantages of the present invention will be apparent from the following description of the presently preferred embodiments of the invention given for the purpose of disclosure.

### BRIEF DESCRIPTION OF THE DRAWINGS

FIGS. 1-3 compare existing corneal topography technology with the invention presented herein.

FIG. 1 is a cross-sectional view of a standard Placido disk corneal topography system, showing 3 corneas that could produce the same reflected image.

FIG. 2 is a cross sectional view of a prismatic corneal topography system showing an optical prism placed between the pattern generator and the eye.

FIG. 3 is the view of a prismatic corneal topography system as seen from the eye being measured. It shows 4 prisms, and the view of the Placido disk rings behind the prism is deviated by refraction in the prisms.

### DETAILED DESCRIPTION OF THE PRESENT INVENTION

For convenience, before further description of the present invention, certain terms employed in the specification, examples and appended claims are collected herein. These definitions should be read in light of the remainder of the disclosure and understood as by a person of skill in the art. Unless defined otherwise, all technical and scientific terms used herein have the same meaning as commonly understood by a person of ordinary skill in the art.

The articles “a” and “an” when used in conjunction with the term “comprising” in the claims and/or the specification, may refer to “one”, but it is also consistent with the meaning of “one or more”, “at least one”, and “one or more than one”. Some embodiments of the invention may consist of or consist essentially of one or more elements, components, method steps, and/or methods of the invention. It is contemplated that any composition, component or method described herein can be implemented with respect to any other composition, component or method described herein.

The term “or” in the claims refers to “and/or” unless explicitly indicated to refer to alternatives only or the alternatives are mutually exclusive, although the disclosure supports a definition that refers to only alternatives and “and/or”.

The terms “comprise” and “comprising” are used in the inclusive, open sense, meaning that additional elements may be included.

The term “including” is used herein to mean “including, but not limited to”. “Including” and “including, but not limited to” are used interchangeably.

As used herein, the terms “optical prism” and “prism” are used interchangeably and refer to an optical element that refracts light.

In one embodiment of this invention, there is provided a corneal topography system for mapping a corneal surface of an eye in a subject, comprising at least one pattern generator; at least one optical prism disposed in optical alignment between the pattern generator and the corneal surface of the eye for which topographical information is desired; a light source is disposed to illuminate the pattern generator; an image sensor disposed in optical alignment with the corneal surface of the eye; and means for electronically transmitting data from the image sensor to an electronic device configured to analyze the data and to display results of the analysis. Further to this embodiment the corneal topography system comprises a focusing lens disposed between the image sensor and the corneal surface.

In both embodiments the pattern generator may comprise alternating dark and light concentric rings. The optical prism may be any shape. It only needs to refract light, causing a deviation in the apparent position of the pattern generator behind it, when viewed from the position of the eye being measured. Ideally, one or two of the edges of the prism will be approximately at right angles to the rings of a Placibo disk pattern generator. In addition, it would also be ideal if the plane of that edge surface or edge surfaces, when extended, would pass through, or close to, the focusing lens.

In another embodiment of the present invention there is provided a prismatic triangulating corneal topography system for mapping a corneal surface, comprising at least one Placido disk; at least one optical prism disposed in optical alignment between the Placido disk and the corneal surface of the eye for which a topographical information is desired; a light source disposed to illuminate the Placido disk; an image sensor disposed in optical alignment with the corneal surface of the eye; and an electronic device with image analysis software tangibly stored therein in electronic communication with the image sensor. Further to this embodiment the corneal topography system comprises an optical lens disposed between the image sensor and the corneal surface.

In both embodiments the optical prism may be for example, a triangular prism, or a cuboid prism. Also, the image sensor may be a charge-coupled device or a complementary metal-oxide semiconductor. In addition, the electronic device may be a desktop computer, a laptop computer, or a smart device.

In yet another embodiment of the present invention there is provided method for mapping a corneal surface of an eye of a subject, comprising positioning an optical prism between a Placido disk and the corneal surface of the eye of the subject in a corneal topography system; illuminating the Placido disk to generate a ring pattern therefrom; acquiring with an image sensor a reflection of the Placido disk from the corneal surface of the eye; transmitting the reflection image from the image sensor to a computer to measure at least one parameter of the corneal surface; and mapping the at least one parameter to produce a corneal topography map of the eye.

Further to this embodiment the method comprises displaying the corneal topography map on the computer. In both embodiments the optical prism may be positioned such that the Placido disk is seen both in the reflection image through the optical prism near an edge of the prism and on the other side of the edge without looking through the prism.

Also in both embodiments at the edge of the optical prism, a deviation of the ring pattern looking through the optical prism compared to the ring pattern at the angle θ looking beside the optical prism provides a line of sight from which the ring pattern is viewed. The amount of deviation seen across the prism edge, of a Placido disk ring edge, can be used to calculate the angle between the corneal reflection point and the physical ring edge point behind the prism edge. Further to this embodiment the method comprises calculating angle α from the reflection image acquired by the image sensor, wherein a light ray at the angle α intersects a light ray with angle θ from beside the Placido ring at the corneal reflection point on the corneal surface. In a further aspect of these embodiments the method comprises measuring the deviation of the ring pattern from light rays at the angle θ beside the prism. In another further aspect the method comprises calculating a surface tangent angle at the corneal surface reflection point from the angle α and the angle θ.

In all embodiments and further aspects thereof, the corneal parameter measured may comprise position, elevation or slope. Also, the optical prism may be any shape, including a triangular prism, or a cuboidal prism. In addition, the image sensor may be a charge-coupled image sensor or a complementary metal-oxide semiconductor image sensor.

Provided herein is a corneal topography system that utilizes at least one optical prism disposed between a means to generate a pattern, such as, but not limited to, a pattern generator, for example, a Placido disk, and the corneal surface of the eye. The corneal topography system may be a prismatic triangulating corneal topography system in which light rays refracted through the prism, light rays bypassing the prism and light rays with angle α calculated from the reflection image enable the corneal reflection point to be triangulated and its position on the corneal surface and the tangential slope to be identified.

The optical prism may be any shape or any type of prism including, but not limited to, a triangular prism, and a cuboidal prism. The prism may be manufactured using any optically transparent material including, but not limited to polycarbonate, pmma, glass, quartz and fluorite.

The prism is positioned with respect to the pattern generator such that a portion of the light source passing from the pattern generator is deviated by refraction through the prism before being incident on the corneal surface, and another portion of the light source passing from the pattern image generator is directly incident on the corneal surface without going through the prism. The prism is preferably oriented such that an edge is approximately perpendicular to the rings in the pattern image generator. Thus, the apparent deviation of the ring enabled by the prism determines a direction from which the ring is viewed. Alternatively, multiple prisms, with their edges aligned perpendicular to the rings, may be used, so that many ring edges cross many prism edges. In this configuration, a large number of reflection points on the corneal surface can have their exact topography parameters calculated accurately, allowing assumptions in the corneal shape to be largely eliminated.

This is advantageous over standard corneal topographers including a Placido disk topographer where a point in the image of the pattern reflected in the corneal surface provides a line in which the corneal reflection point must be on, but cannot differentiate between a flat corneal surface further away or a steeper corneal surface closer. Thus, the present invention provides a unique solution for the reflection point on the cornea, both for position, including elevation, and surface slope. In addition, these prisms allow a quick and simple point and shoot measurement process, that does not need any special alignment.

The light source illuminates the image pattern generator. The light source may be any light source suitable for corneal topography as is known and standard in the art. For example, LEDs, Fluorescent bulbs or sources, and filament bulbs. Because of the prisms, monochrome light may be more accurate than a broad-spectrum source.

The corneal topography system comprises an image sensor that captures an image of the pattern reflected from the corneal surface of the eye. Any commercially available stand-alone image sensors comprised in a digital camera or a smart device or, image sensors integrated with a computer for image analysis may be used for this purpose Examples of such image sensors include a charge-coupled device (CCD) and an active-pixel sensor (CMOS sensor).

The corneal topography system comprises a means for focusing an image of the pattern reflected from the corneal surface onto the image sensor before the image is digitally captured by the image sensor. For example, a focusing lens or an optical lens is disposed in optical alignment between the image sensor and the corneal surface. Any commercially available lens may be utilized to serve as the focusing lens or the optical lens. The lens may be made using any optically transparent material including, but not limited to, glass, quartz and plastic. A pin-hole lens would also work.

The corneal topography system is electronically linked to means for electronically transmitting data from the image sensor to an electronic device configured to analyze the data and to display results of the analysis. The means may comprise any type of commercially available electronic device with a processor, a memory, and display may be used. These devices include, but not limited to, a desktop computer, a laptop computer, a hand-held computer or tablet, and a smart device. The electronic device is in wired or wireless communication with the image sensor. The electronic device tangibly stores that software, firmware and/or algorithms suitable for analysis of the captured reflection image as is known and standard in the art.

Also provided is a method for mapping a corneal surface of an eye of a subject that utilizes the corneal topography systems described herein. Particularly, the optical prism is placed between a Placido disk and the corneal surface of the eye. The subject for which topographical information of the corneal surface of the eye is desired, has the eye positioned near the image plane of the image sensor. The Placido disk is imaged by reflection from the corneal surface of that eye. Parts of the Placido disk image are seen refracted in the optical prism before being reflected off the cornea, and parts are viewed directly reflected off the cornea. In the Placido disk image it is possible to see the amount of deviation of the image of the Placido disk that was caused by the prism, by comparing the ring images across the edge of the prism. The amount of deviation allows angle θ to be calculated. Angle α can be calculated from the position of the non-deviated ring on the image sensor. Using the known physical parameters of the corneal topography device, angle α and angle θ can be used to determine the position of the reflection point of the cornea, and the angle half way between these two angles provides the angle of the line that is normal to the corneal surface at that point. The slope at that point is then easily calculated.

Particularly, embodiments of the present invention are better illustrated with reference to the Figure(s), however, such reference is not meant to limit the present invention in any fashion. The embodiments and variations described in detail herein are to be interpreted by the appended claims and equivalents thereof.

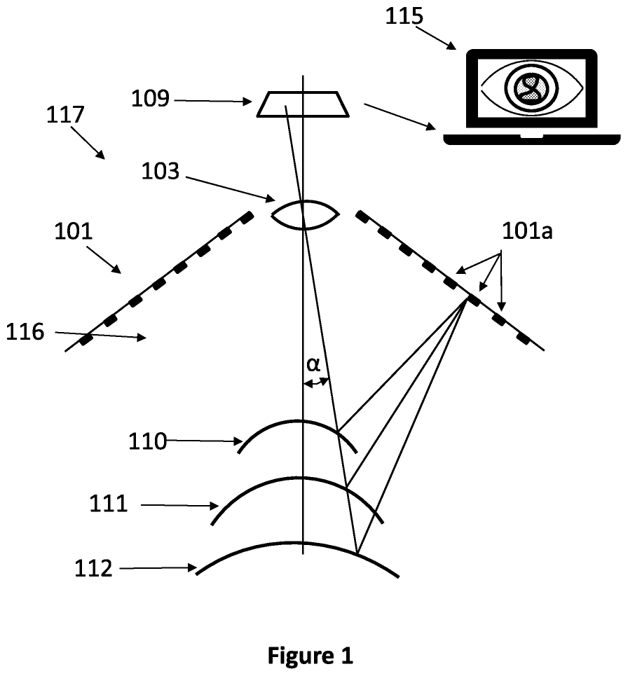

FIG. 1 shows a cross-section of a corneal topography system 117 as known in the art. The system consists of a cone-shaped pattern generator 116 that generates an image reflected in a corneal surface 110, 111, 112, a lens 103 that focuses the reflected light from the corneal surface to a charge-coupled device (CCD) 109, which serves as the image sensor. The image captured by the charge-coupled device is transmitted to a computer 115 for analysis. This system relies on the assumptions of corneal shape for applying the calculations. Without prior knowledge however on how far the corneal surface 110, 111, 112 is from the topography device, there are many possible solutions for corneal topography including, a steep corneal surface 110 closer to the topographer, a flat corneal surface 112 further away from the device and a medium curvature corneal surface 111 in between.

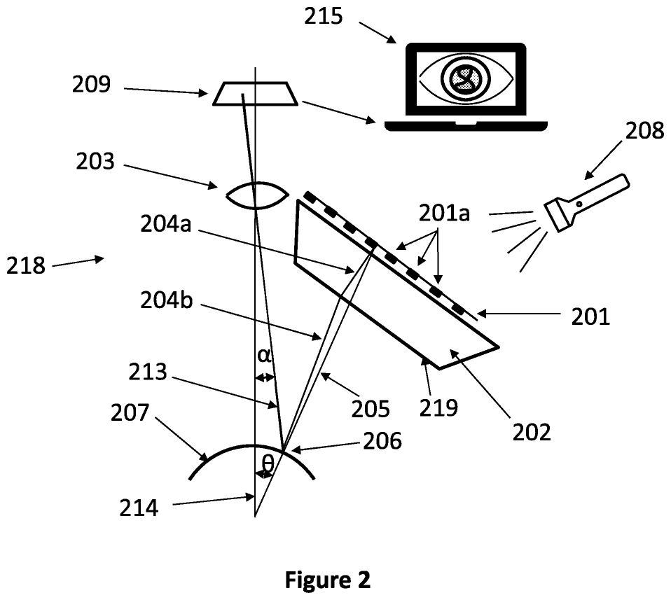

FIG. 2 is one side of a cross-sectional view of the prism based corneal topography system 218 along an edge 219 of the prism 202 showing an illumination source 208, a Placido disk pattern generator 201 with a plurality of rings 201a disposed thereon and a lens 203. A ring image reflected off the corneal surface 207 passes through the lens and is captured by a charge-coupled device (CCD) 209 used as the image sensor. The CCD image captured by the charge-coupled device is transmitted to a computer 215 for analysis. From the image, a first angle α is measured between the topography system's optical axis 214 and where a ring edge is detected on the image sensor 209. A second angle θ is determined from the difference between the light path passing through the prism on one edge of the prism 204a and 204b and the light path 205 not passing through the prism on the other edge of the prism. Since only light rays at angle θ along the edge of the prism will have the measured deviation when moving through the prism, the deviation can also be measured from the CCD image.

The point of intersection of the light path at angle θ from the ring edge at 201a, and the light path at angle α through the lens 203 from the image sensor 209, is the corneal surface reflection point 206. The two angles α and θ also enable the surface tangent angle at that surface reflection point to be easily calculated.

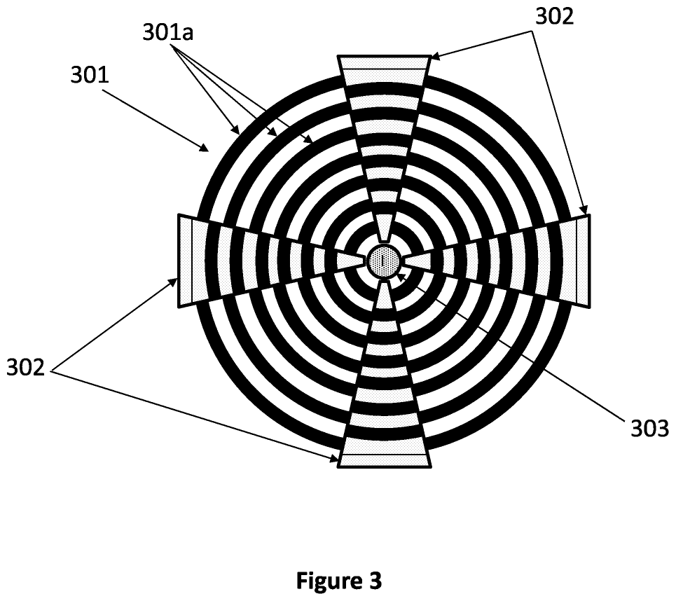

FIG. 3 shows a view of the prism based corneal topography system, seen from the direction of the eye being measured. This example shows four prisms 302. Behind the prisms is a pattern generator 301 with a plurality of rings 301a disposed thereon. Lens 303 can be seen in the center. The rings 301a appear deviated in position, when viewed through the prisms 302.

The present invention is well adapted to attain the ends and advantages mentioned as well as those that are inherent therein. The particular embodiments disclosed above are illustrative only, as the present invention may be modified and practiced in different but equivalent manners apparent to those skilled in the art having the benefit of the teachings herein. Furthermore, no limitations are intended to the details of construction or design herein shown, other than as described in the claims below. It is therefore evident that the particular illustrative embodiments disclosed above may be altered or modified and all such variations are considered within the scope of the present invention. The terms in the claims have their plain, ordinary meaning unless otherwise explicitly and clearly defined by the patentee.

### The Following References are Cited Herein:

1. van Saarloos, P P and Constable, I J. (1991) Optometry and Vision Science, 68 (12): 960-965.

What is claimed is:

1. A corneal topography system (218) for mapping a corneal surface (207) of an eye in a subject, comprising:
at least one pattern generator (201);
at least one optical prism (202) disposed in optical alignment between the pattern generator (201) and the corneal surface (207) of the eye for which topographical information is desired;
a light source (208) disposed in position to illuminate the pattern generator (201);
an image sensor (209) disposed in optical alignment with the corneal surface (207) of the eye; and
means for electronically transmitting data from the image sensor (209) to an electronic device (215) configured to analyze the data and to display results of the analysis.

2. The corneal topography system (218) of claim 1, further comprising a focusing lens (203) disposed between the image sensor (209) and the corneal surface (207) of the eye.

3. The corneal topography system (218) of claim 1, wherein the pattern generator (201) comprises alternating dark and light concentric rings (201a).

4. The corneal topography system (218) of claim 1, wherein the image sensor (209) is a charge-coupled device or a complementary metal-oxide semiconductor.

5. A prismatic triangulating corneal topography system (218) for mapping a corneal surface (207) of an eye, comprising:
at least one pattern generator (201);
at least one prism (202) disposed in optical alignment between the pattern generator (201) and the corneal surface (207) of the eye for which topographical information is desired;
a light source (208) positioned to illuminate the pattern generator (201);
an image sensor (209) disposed in optical alignment with the corneal surface (207) of the eye;
an imaging system to project the pattern generator image reflected in the corneal surface (207) onto the image sensor (209); and
an electronic device (215) comprising image analysis software tangibly stored therein in electronic communication with the image sensor (209).

6. The prismatic triangulating corneal topography system (218) of claim 5, wherein the image sensor (209) is a charge-coupled device or a complementary metal-oxide semiconductor.

7. The prismatic triangulating corneal topography system (218) of claim 5, wherein the electronic device (215) is a desktop computer, a laptop computer, or a smart device.

8. A method for mapping a corneal surface (207) of an eye of a subject, comprising:
positioning at least one optical prism (202) between a pattern generator (201) and the corneal surface (207) of the eye of the subject in a corneal topography system (218);
illuminating the pattern generator (201) so that it can be seen reflected in the corneal surface, with part of the pattern generator (201) seen through the prism (202) and part seen directly around the edge of the prism (202);
using an optical system to project the reflected image of the pattern generator (201) from the corneal surface (207) onto an image sensor (209);
acquiring an image from the image sensor (209) and transmitting that reflection image from the image sensor (209) to a computer (215) to measure at least one parameter of the corneal surface (207); and
mapping the at least one parameter to produce a corneal topography map of the eye.

9. The method of claim 8, where the pattern generator (201) is a Placido disk.

10. The method of claim 8, wherein the optical prism (202) is positioned such that the pattern generator (201) is seen in the reflection image through the optical prism (202) and beside the optical prism (202) across at least two edges of the prism (202).

11. The method of claim 8, wherein, at the edge of the optical prism (202), a deviation of the pattern generator (201) looking through the optical prism (202) compared to the pattern generator (201) looking beside the optical prism at angle θ provides a line of sight from which the pattern generator (201) is viewed;
the angle θ to be calculated;
calculating angle α from the reflection image acquired by the image sensor (209);
wherein the light ray (213) at angle α intersects a light ray (205) with angle θ from the pattern generator at the corneal reflection point (206) on the corneal surface (207).

12. The method of claim 11, further comprising measuring the deviation of the pattern generator (201) from light rays (205) at the angle θ beside the prism (202).

13. The method of claim 11, further comprising calculating a surface tangent angle at the corneal surface reflection point (206) from the angle α and the angle θ.

14. The method of claim 8, wherein the parameter comprises position, elevation or slope.

15. The method of claim 8, wherein the image sensor (209) is a charge-coupled image sensor or a complementary metal-oxide semiconductor image sensor.
